(19) United States
(12) Patent Application Publication
(10) Pub. No.: US 2011/0218617 A1
(43) Pub. Date: Sep. 8, 2011

(54) ENDOLUMINAL VASCULAR PROSTHESIS

(75) Inventors: Jonathon Nguyen, Garden Grove, CA (US); Jacqueline Macias, South Gate, CA (US); Elbert Tzeng, Irvine, CA (US); Stefan Schreck, Fallbrook, CA (US)

(73) Assignee: ENDOLOGIX, INC., Irvine, CA (US)

(21) Appl. No.: 13/039,157

(22) Filed: Mar. 2, 2011

Related U.S. Application Data
(60) Provisional application No. 61/309,797, filed on Mar. 2, 2010.

(51) Int. Cl. A61F 2/82

(52) U.S. Cl. 623/1.35

(57) ABSTRACT

Some embodiments of an endoluminal prosthesis comprise a graft having a first end and a second end, a first stent positioned at a first end of the graft, the first stent comprising a plurality of proximal apices and a plurality of distal apices, a second stent positioned axially adjacent to the first stent comprising a plurality of proximal apices positioned at a first end of the second stent. In some embodiments, the first stent can be partially covered by the graft such that the proximal apices of the first stent are not covered by the graft. The distal apices of the first stent can be positioned approximately on a first or a second plane offset from the first plane. The second stent can be positioned relative to the first stent such that the plurality of proximal apices of the second stent are spaced apart from the plurality of distal apices of the first stent. Further, one or more of the proximal apices of the second stent can be positioned approximately on a third or a fourth plane. The distal apices of the first stent can be circumferentially offset from the proximal apices of the second stent.

### PRIORITY INFORMATION AND INCORPORATION BY REFERENCE

This application claims priority benefit of U.S. Provisional Application 61/309,797 (titled “ENDOLUMINAL VASCULAR PROSTHESIS”), filed Mar. 2, 2010, which application is hereby incorporated by reference in its entirety as if fully set forth herein. The benefit of priority is claimed under the appropriate legal basis including, without limitation, under 35 U.S.C. §119(e).

Additionally, U.S. Pat. No. 6,733,523, filed on Jun. 26, 2001, U.S. Pat. No. 6,077,296, filed on Mar. 4, 1998, and U.S. Provisional Patent Application No. 61/231,898, filed on Aug. 6, 2009 are hereby incorporated by reference in their entireties as if fully set forth herein.

### TECHNICAL FIELD

The present disclosure relates to endoluminal vascular prostheses, and, in certain embodiments, to endoluminal vascular prostheses for use in the treatment of abdominal aortic aneurysms.

### BACKGROUND OF THE DISCLOSURE

An abdominal aortic aneurysm is a sac caused by an abnormal dilation of the wall of the aorta, a major artery of the body, as it passes through the abdomen. The abdomen is that portion of the body that lies between the thorax and the pelvis. It contains a cavity, known as the abdominal cavity, separated by the diaphragm from the thoracic cavity and lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries.

The aneurysm usually arises in the infrarenal portion of the diseased aorta, for example, below the kidneys. When left untreated, the aneurysm may eventually cause rupture of the sac with ensuing fatal hemorrhaging in a very short time. High mortality associated with the rupture led initially to transabdominal surgical repair of abdominal aortic aneurysms. Surgery involving the abdominal wall, however, is a major undertaking with associated high risks. There is considerable mortality and morbidity associated with this magnitude of surgical intervention, which in essence involves replacing the diseased and aneurysmal segment of blood vessel with a prosthetic device which typically is a synthetic tube, or graft, usually fabricated of polyester, urethane, Dacron®, Teflon®, or other suitable material.

To perform the surgical procedure requires exposure of the aorta through an abdominal incision which can extend from the rib cage to the pubis. The aorta must typically be closed both above and below the aneurysm, so that the aneurysm can then be opened and the thrombus, or blood clot, and arteriosclerotic debris removed. Small arterial branches from the back wall of the aorta are tied off. The Dacron® tube, or graft, of approximately the same size of the normal aorta is sutured in place, thereby replacing the aneurysm. Blood flow is then reestablished through the graft. It is necessary to move the intestines in order to get to the back wall of the abdomen prior to clamping off the aorta.

If the surgery is performed prior to rupturing of the abdominal aortic aneurysm, the survival rate of treated patients is markedly higher than if the surgery is performed after the aneurysm ruptures, although the mortality rate is still quite high. If the surgery is performed prior to the aneurysm rupturing, the mortality rate is typically slightly less than 10%. Conventional surgery performed after the rupture of the aneurysm is significantly higher, one study reporting a mortality rate of 66.5%. Although abdominal aortic aneurysms can be detected from routine examinations, the patient does not experience any pain from the condition. Thus, if the patient is not receiving routine examinations, it is possible that the aneurysm will progress to the rupture stage, wherein the mortality rates are significantly higher.

Disadvantages associated with the conventional, prior art surgery, in addition to the high mortality rate include the extended recovery period associated with such surgery; difficulties in suturing the graft, or tube, to the aorta; the loss of the existing aorta wall and thrombosis to support and reinforce the graft; the unsuitability of the surgery for many patients having abdominal aortic aneurysms; and the problems associated with performing the surgery on an emergency basis after the aneurysm has ruptured. A patient can expect to spend from one to two weeks in the hospital after the surgery, a major portion of which is spent in the intensive care unit, and a convalescence period at home from two to three months, particularly if the patient has other illnesses such as heart, lung, liver, and/or kidney disease, in which case the hospital stay is also lengthened. Since the graft must typically be secured, or sutured, to the remaining portion of the aorta, it is many times difficult to perform the suturing step because the thrombosis present on the remaining portion of the aorta, and that remaining portion of the aorta wall may be friable, or easily crumbled.

Since many patients having abdominal aortic aneurysms have other chronic illnesses, such as heart, lung, liver, and/or kidney disease, coupled with the fact that many of these patients are older, the average age being approximately 67 years old, these patients are not ideal candidates for such major surgery.

More recently, a significantly less invasive clinical approach to aneurysm repair, known as endovascular grafting, has been developed. Parodi, et al. provide one of the first clinical descriptions of this therapy. Parodi, J. C., et al., “Transfemoral Intraluminal Graft Implantation for Abdominal Aortic Aneurysms,” 5 Annals of Vascular Surgery 491 (1991). Endovascular grafting involves the transluminal placement of a prosthetic arterial graft in the endoluminal position (within the lumen of the artery). By this method, the graft is attached to the internal surface of an arterial wall by means of attachment devices (expandable stents), typically one above the aneurysm and a second stent below the aneurysm.

In general, transluminally implantable prostheses, or grafts, adapted for use in the abdominal aorta comprise a tubular wire cage, or stent, surrounded by a tubular PTFE or Dacron sleeve. Stents can permit fixation of a graft to the internal surface of an arterial wall without sewing or an open surgical procedure. Both balloon expandable and self expandable support structures have been proposed. Expansion of radially expandable stents is conventionally accomplished by dilating a balloon at the distal end of a balloon catheter. In U.S. Pat. No. 4,776,337, for example, Palmaz describes a balloon-expandable stent for endovascular treatments. Self-expanding stents have been described, for example, in U.S. Pat. No. 4,655,771 to Wallsten. Endovascular grafts adapted to treat both straight segment and bifurcation aneurysms have also been proposed.

In certain conditions, the diseased region of the blood vessels can extend across branch vessels. The blood flow into these branch vessels is critical for the perfusion of the peripheral regions of the body and vital organs. Many arteries branch off the aorta. For example, the carotid arteries supply blood into the brain, the renal arteries supply blood into the kidneys, the superior mesenteric artery (“SMA”) supplies the pancreas, the hypogastric arteries supply blood to the reproductive organs, and the subclavian arteries supply blood to the arms. When the aorta is diseased, the branch vessels may also be affected. Thoracic aortic aneurysms may involve the subclavian and carotid arteries, abdominal aneurysms may involve the SMA, renal and hypogastric arteries. Aortic dissections may involve all branch vessels mentioned above. When this occurs, it may be detrimental to implant a conventional tubular graft in this location of the aorta or the blood vessel, since such a graft may obstruct the flow of blood from the aorta into the branches. Additionally, properly located deployment of prosthetic grafts adjacent branches in the aorta present risks of flow obstruction in the branch vessels because identifying the distal ends of the grafts can be difficult under fluoroscopy.

Grafts and graft systems are typically used to treat aneurysms in the aorta or in other blood vessels. These grafts can be positioned within the aorta or other blood vessels at the location of an aneurysm and, generally speaking, can provide a synthetic vessel wall that channels the flow of blood through the diseased portion of the blood vessel. As such, the grafts are typically fluid impermeable so that no blood can flow through the walls of the graft. Rather, the blood is channeled through the central passageway defined by the graft. Leakage of blood flow between the graft and the blood vessel tissue can risk further deterioration of the diseased aneurysm.

The stent graft system isolates the aneurysms from the blood pressure of the aorta preventing rupture of the aneurysm. In order for the stent graft to isolate the aneurysm, it has to provide a hermetic seal proximal and distal to the aneurysm. This can be specifically challenging in infrarenal aneurysms where the proximal seal zone below the renal arteries is often short, angulated, and calcified.

Accordingly, there is a need to accurately place endoluminal prostheses in the aorta without obstructing critical branch vessels in a minimally invasive manner. There is a further need to provide a sufficient seal between the graft and the vessel wall, even in challenging anatomical situations. The embodiments of the endoluminal prostheses disclosed herein provide a solution to the problems described above.

### SUMMARY OF SOME EXEMPLIFYING EMBODIMENTS

Some embodiments comprise an endoluminal prosthesis that can include a first support having a proximal end and a distal end and a second support having a proximal end and a distal end. The second support can be located closer to a proximal end of the prosthesis as compared to the first support. A cover at least substantially covers the first support and at least a portion of the second support. At least a portion of each of the first and second supports can be coupled to the cover. The second support comprises distal apices that can be longitudinally and/or circumferentially offset as compared to proximal apices of the first support.

Some embodiments are directed to a method of deploying an endoluminal prosthesis in vasculature. The method includes inserting an endoluminal prosthesis within the vasculature. The endoluminal graft has a first support and a second support coupled to a sleeve. The second support can be coupled to a distal end of the sleeve and extends longitudinally beyond the distal end of the sleeve. The endoluminal prosthesis can be positioned adjacent a vascular branch blood vessel by locating a distal end of the first support adjacent the branch opening, the distal end of the first support coinciding with the distal end of the sleeve. The endoluminal prosthesis can be expanded such that the second support spans the vessel branch to make contact with the vessel wall on the opposing side of the vessel branch.

Some embodiments recite an endoluminal prosthesis comprising a graft having a first end and a second end, a first stent positioned at a first end of the graft, the first stent comprising a plurality of proximal apices and a plurality of distal apices, and a second stent positioned axially adjacent to the first stent comprising a plurality of proximal apices positioned at a first end of the second stent. In some embodiments, the first stent can be partially covered by the graft such that the proximal apices of the first stent are not covered by the graft. Further, one or more of the distal apices of the first stent can be positioned approximately on a first plane that can be perpendicular to a longitudinal axis of the first stent, and one or more of the distal apices of the first stent can be positioned approximately on a second plane that can be perpendicular to a longitudinal axis of the first stent, the second plane being offset from the first plane.

In some embodiments, the second stent can be positioned relative to the first stent such that the plurality of proximal apices of the second stent can be spaced apart from the plurality of distal apices of the first stent. One or more of the proximal apices of the second stent can be approximately positioned on a third plane that can be perpendicular to a longitudinal axis of the second stent, the third plane being offset from the first and second planes. One or more of the proximal apices of the second stent can be positioned approximately on a fourth plane that can be perpendicular to a longitudinal axis of the second stent, the fourth plane being offset from the first, second, and third planes. Further, the distal apices of the first stent can be circumferentially offset from the proximal apices of the second stent.

Some embodiments recite an endoluminal prosthesis comprising a first support having a proximal end and a distal end, a second support spaced apart from the first support having a proximal end and a distal end, the second support being located closer to a distal end of the prosthesis as compared to the first support, and a cover that at least substantially covers the second support and at least a portion of the first support. In some embodiments, at least a portion of each of the first and second supports can be attached to the cover. The first support can comprise distal apices that can be longitudinally and circumferentially offset as compared to proximal apices of the second support. The distal apices can be interdigitated relative to the proximal apices of the second support.

Some arrangements recite a method of deploying an endoluminal prosthesis in vasculature, comprising inserting an endoluminal prosthesis within the vasculature, positioning the endoluminal prosthesis adjacent a vascular branch blood vessel by locating a proximal end of the second support adjacent the branch opening, and expanding the endoluminal prosthesis. Some embodiments of the endoluminal graft can have a first support and a second support coupled to a sleeve, wherein the first support can be coupled to a proximal end of the sleeve and can extend longitudinally beyond the proximal end of the sleeve. In any of the embodiments disclosed herein, the first or proximal support can be completely or nearly completely covered by the sleeve. In some embodiments, the proximal end of the second support can be positioned approximately coincident with the proximal end of the sleeve.

The first support can span the vessel branch to make contact with the vessel wall on the opposing side of the vessel branch. One or more distal apices of the first support can be longitudinally offset from other distal apices of the first support, and one or more distal apices of the second support can be longitudinally offset from other distal apices of the second support. Further, without limitation, each of the distal apices of the second support can be circumferentially offset from each of the proximal apices of the first support. In any of the embodiments disclosed herein, one or more of the distal apices of the second support can be approximately circumferentially aligned with each of the proximal apices of the first support.

### BRIEF DESCRIPTION OF THE DRAWINGS

FIG. 1 is a partial cross-sectional view of a main graft body and an extension graft body of an endoluminal prosthetic device in accordance with an embodiment that is deployed in the desired position within a patient's vasculature.

FIG. 2 is an expanded side view of a main graft body and an extension graft body of the endoluminal prosthetic device of FIG. 1.

FIG. 3 is an expanded side view of the extension graft body of the endoluminal prosthetic device of FIG. 1.

FIG. 4 is an expanded side view of the main graft body of the endoluminal prosthetic device of FIG. 1.

FIG. 5A is a front view of an embodiment of a support link of the main graft body and the extension graft body of the endoluminal prosthetic device of FIG. 1.

FIG. 5B is a side view of the support link of FIG. 5A.

FIG. 5C is a front view of an embodiment of a support link of the main graft body and the extension graft body of the endoluminal prosthetic device of FIG. 1.

FIG. 5D is a side view of the support link of FIG. 6A.

FIG. 6A is a schematic side view of the internal surface of the circumference of a distal portion of the main graft body, laid out flat, of an embodiment of an endoluminal prosthetic device.

FIG. 6B is a schematic side view of the internal surface of the circumference of a distal portion of the extension graft body, laid flat, of the endoluminal prosthetic device of FIG. 1.

FIG. 7 is side views of an internal surface of a distal portion of the extension graft body of the endoluminal prosthetic device of FIG. 1.

FIG. 8 is a side view of a portion of the extension graft body of the endoluminal prosthetic device of FIG. 1.

FIGS. 9-11 are schematic side views of the internal surface of the circumference of a distal portion of the extension graft body, laid flat, of the endoluminal prosthetic device of FIG. 1

### DETAILED DESCRIPTION OF SOME EXEMPLIFYING EMBODIMENTS

The following detailed description is now directed to certain specific embodiments of the disclosure. In this description, reference is made to the drawings wherein like parts are designated with like numerals throughout the description and the drawings.

Certain embodiments described herein are directed to systems, methods, and apparatuses to treat lesions, aneurysms, or other defects in the aorta, including, but not limited to, the thoracic, ascending, and abdominal aorta, to name a few. However, the systems, methods, and apparatuses may have application to other vessels or areas of the body, or to other fields, and such additional applications are intended to form a part of this disclosure. For example, it will be appreciated that the systems, methods, and apparatuses may have application to the treatment of blood vessels in animals. In short, the embodiments and/or aspects of the endoluminal prosthesis systems, methods, and apparatuses described herein can be applied to other parts of the body or may have other applications apart from the treatment of the thoracic, ascending, and abdominal aorta. And, while specific embodiments may be described herein with regard to particular portions of the aorta, it is to be understood that the embodiments described can be adapted for use in other portions of the aorta or other portions of the body and are not limited to the aortic portions described.

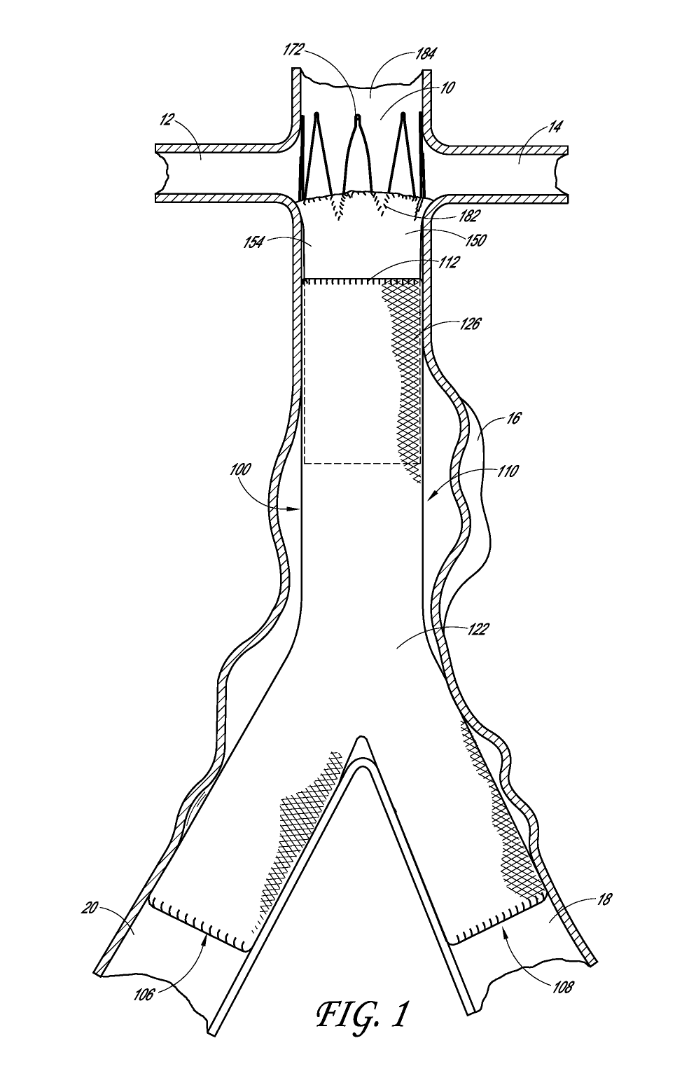

FIG. 1 is a partial cross-sectional schematic view of an endoluminal prosthetic device 100 in accordance with an embodiment that is deployed in the desired position within a patient's vasculature. The schematically illustrated vasculature is the aorta 10, which can have a vessel branch including the right renal artery 12 and the left renal artery 14. Additional vessels (e.g., second lumbar, testicular, inferior mesenteric, middle sacral) have been omitted for simplification. Vascular aneurysms 16 of the aorta 10 are generally located below the renal arteries 12, 14.

FIG. 1 illustrates an embodiment of the main graft 110 and an embodiment of a stent or stent graft 150 deployed in the aorta 10. In some embodiments, the stent 150, which can be a suprarenal stent extension, can be deployed within a patient's vasculature independent of (i.e., without) a main graft 110. The main graft 110 can be a self-expanding bifurcated stent graft, as illustrated, or can be a tubular non-bifurcated stent graft that can be self-expanding, mechanically expandable, or otherwise. In some embodiments, the stent 150 or any other stent in this disclosure can have a tubular body (as illustrated) or can be bifurcated or otherwise configured to conform to any suitable luminal geometry. The stent 150 or any other stent in this disclosure can be self-expanding, mechanically expandable, or otherwise.

In the illustrated embodiment, the main graft body 110 can be a bifurcated graft, as illustrated, or can be a tubular or non-bifurcated graft. Accordingly, the main graft 110 can have a first bifurcated branch 106 and a second bifurcated branch 108 that can be coupled to the proximal end 114, adjacent each other, and extend into the right common iliac artery 20 and the left common iliac artery 18, respectively.

In some embodiments, though not required, the main graft 110 can be a bifurcated stent graft, extending into the left common iliac artery 18 and the right common iliac artery 20. As illustrated in FIG. 1, the main graft 110 can form an artificial lumen within the diseased portion of the aorta 10 (e.g., the aneurysm) by spanning the length of the aneurysm 16. In some embodiments, the main graft 110 can extend beyond the proximal and distal ends of the aneurysm 16 to mitigate the exposure of the diseased portion of the vessel to the blood flow and blood pressure. Thus, the main graft body 110 can isolate the aneurysm from the pressure of the blood flowing through the aorta, preventing rupture of the aneurysm.

In some embodiments, the main graft 110 and the stent 150 and other components of the device 100 can be formed or deployed as disclosed in U.S. Pat. No. 6,733,523, filed on Jun. 26, 2001, U.S. Pat. No. 6,077,296, filed on Mar. 4, 1998, and/or U.S. Provisional Patent Application No. 61/231,898, filed on Aug. 6, 2009. The entire disclosures of each of U.S. Pat. Nos. 6,733,523 and 6,077,296, and U.S. Provisional Patent Application No. 61/231,898 are hereby incorporated by reference as if fully set forth herein.

As depicted in FIG. 1, the main graft 110 can have a wire support 124 (which can be, but is not required to be, self-expanding) and a sleeve 126. The support 124 and sleeve 126 are more readily shown in the exploded view of FIG. 4. The support 124 can have a substantially tubular shaped wire body extending from a proximal end 112 spanning to a distal end 114 adjacent the left and right common iliac arteries 18, 20. As used herein with reference to the device 100, the term proximal refers to the upstream end or portion of the device 100, while distal refers to the downstream end or portion of the device 100.

The main graft 110 can have a proximal portion 119 that can have a first proximal segment 116 and a second proximal segment 118 of the support 124. The support 124 can have additional segments that can couple together and extend distally to define the full length of the support. The main graft 110 further can have an intermediate portion 120 and a distal portion 122 that define the long straight portion of the main graft 110 and support 124. The first and second proximal segments 116, 118 can be formed from a single continuous wire. In some embodiments, the support 124 can be made up of more than one wire.

The sleeve 126 can be coupled to the support 124 in a concentric manner on an outside surface of the tubular shaped support 124. In some embodiments, the sleeve 126 can be supported on an inside surface of the support 124, or alternatively, at least a portion of the sleeve 126 can be supported on the inside surface and another portion on the outside surface. The sleeve 126 can be coupled to the support 124 by stitching via sutures, adhesives, laser bonding, clips, or by any other suitable means. The bifurcated branch portions of the sleeve 126 can be integrally formed with the long straight section of the sleeve, or can be formed separately and thereafter affixed to the straight section via stitching or by any suitable means, so as to define a single piece sleeve material.

The polymeric sleeve 126 can be made out of a variety of synthetic polymeric materials, including PTFE, ePTFE, PE, PET, urethane, Dacron, nylon, polyester, woven material, or the like, or any combination thereof. The sleeve material can be configured to exhibit limited elasticity upon expansion to the desired approximate diameter of the expanded support 124 when deployed within the aorta 10. The sleeve can have a wall thickness of approximately 0.0015 in., or from approximately 0.001 in to approximately 0.003 inches or more.

As illustrated in FIG. 1, in some embodiments, the main graft 110 can be deployed so that the proximal end thereof is in an infrarenal position below the renal arteries. Thereafter, in some embodiments, the stent 150 can be deployed proximal to the main graft 110. The main graft 110 and stent 150 can be deployed by any suitable method, including without limitation, being deployed percutaneously through a patient's femoral artery. In some embodiments, as is illustrated in FIG. 1, a proximal portion of the stent 150 (i.e., the upstream end portion of the stent 150) can span or extend distally beyond the ostium of each of the renal arteries 12, 14 such that a proximal end portion of the stent 150 contacts the wall of the aorta proximal or above the renal ostium. In some embodiments, not illustrated, the main stent graft 110 can have some or all of the features of the stent 150 or any other stent disclosed herein or incorporated by reference herein.

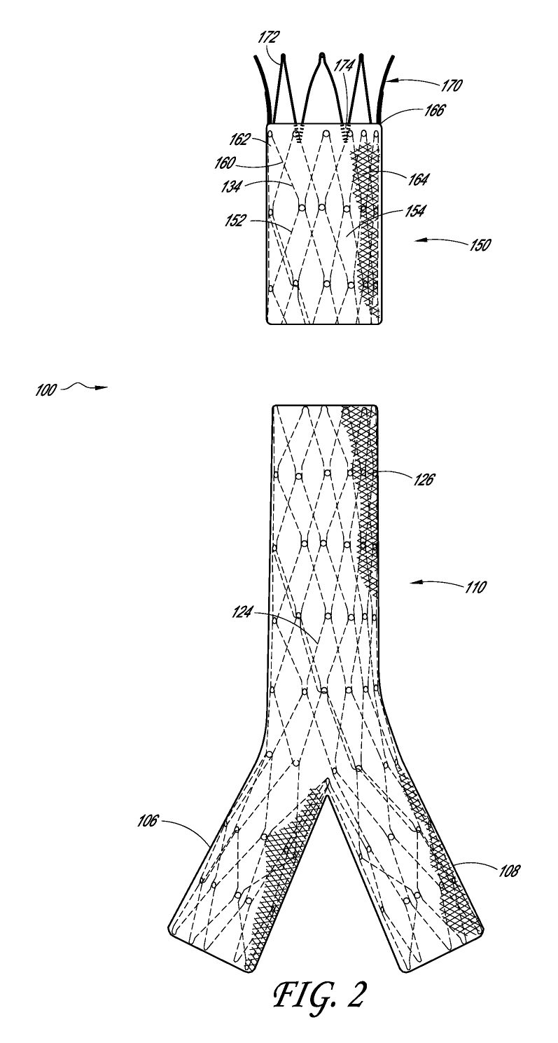

With reference to FIGS. 1-3 and 6-11, the stent 150 can provide added longitudinal length to the endoluminal prosthesis 100, enabling a medical practitioner to support a wide range of anatomical geometries. For example, the stent 150 can provide additional structural support to the renal portion of the aorta 10 and can improve the sealing function of the proximal or distal end portion(s) of the endoluminal prosthesis 100 by providing additional, staggered contact points against the wall of the aorta 10.

The stent 150 can have an infrarenal configuration (not shown) and a suprarenal configuration (as illustrated in FIG. 1). The infrarenal configuration can terminate distal to, or downstream of the ostium of the renal arteries 12, 14. The infrarenal configuration can have a graft 154 covering substantially the full length of the underlying wire body support 152. In some embodiments, the underlying proximal end of the wire body support 152 (also referred to herein as a first stent or a stent segment) or any other portion thereof, can be exposed in the infrarenal configuration.

The suprarenal configuration stent 150, illustrated in FIG. 1, can have an uncovered proximal end portion that substantially spans the ostium of the renal arteries 12, 14. In this configuration, the distal portion of the stent 150 can be uncovered to facilitate blood flow into the renal arteries 12, 14. Further details of the suprarenal configuration stent 150 are described below.

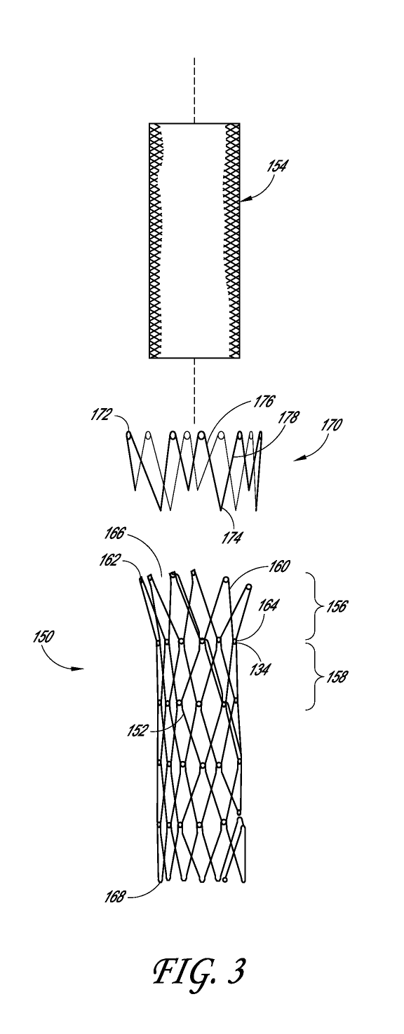

As illustrated in FIG. 3, the wire body support 152 can have the proximal end 166 and a distal end 168. Upon being deployed, the proximal end 166 can extend adjacent to or just below the renal arteries 12, 14, and the distal end 168 can be positioned within an end portion of the main graft 110, as illustrated in FIG. 1. The wire body support 152 can have a first proximal segment (or infrarenal segment) 156 adjacent to the proximal end 166, and a second proximal segment 158 adjacent to the first proximal segment. Additionally, the stent 150 can have an additional proximal segment (or suprarenal segment) 170 that is positioned adjacent to the segment 156. As illustrated in FIG. 1, in some embodiments, the segment 170 can have a proximal end 184 that extends above the renal arteries 12, 14 into the suprarenal portion of the aorta 10. Thus, the extension graft 150, when fully deployed, can extend from within the intermediate portion 120 of the main graft 110 to the suprarenal portion of the aorta (i.e., above the renal arteries 12, 14).

With reference to FIG. 3, in some embodiments, the first proximal segment 156 of the wire body support 152 can have a plurality of struts 160, a plurality of proximal apices 162, and a plurality of distal apices 164. The proximal apices 162 can define the proximal end of the first proximal segment 156, and the distal apices 164 define the distal end of the first proximal segment 156. A plurality of struts 160 can connect adjacent proximal and distal apices 162, 164. As illustrated, the first proximal segment 156 can have a proximal bend or apex 162, connected via a distally directed strut 160 to a corresponding distal bend or apex 164, which in turn can be connected via a proximally directed second strut 160 to a second proximal apex 162. This configuration can continue so as to form a generally zig-zag pattern around the circumference of the stent 150. In some embodiments, as illustrated, the distal bends 164 can be connected to proximal apices or bends of an adjacent stent segment (e.g., stent segment 158) positioned distal to the stent segment 156. In some embodiments, the distal bends 164 can be spaced apart from the proximal apices or bends of the adjacent stent segment (e.g., stent segment 158) positioned distal to the stent segment 156, the stent segments 156, 158 being sutured or otherwise attached to or otherwise supported by a graft 154 for support.

A cover or graft 154 can cover a portion of the wire body support 152 of the stent 150. As described above, the proximal end of the stent 150 can be uncovered to avoid obstructing the renal arteries. For example, the segment 170 can be substantially uncovered to avoid obstructing the renal arteries. Thus, the first proximal segment 156, or infrarenal segment, can be configured to be substantially or completely covered by the graft 154, whereas the segment 170, or suprarenal segment, can be configured to be only partially covered by the graft 154.

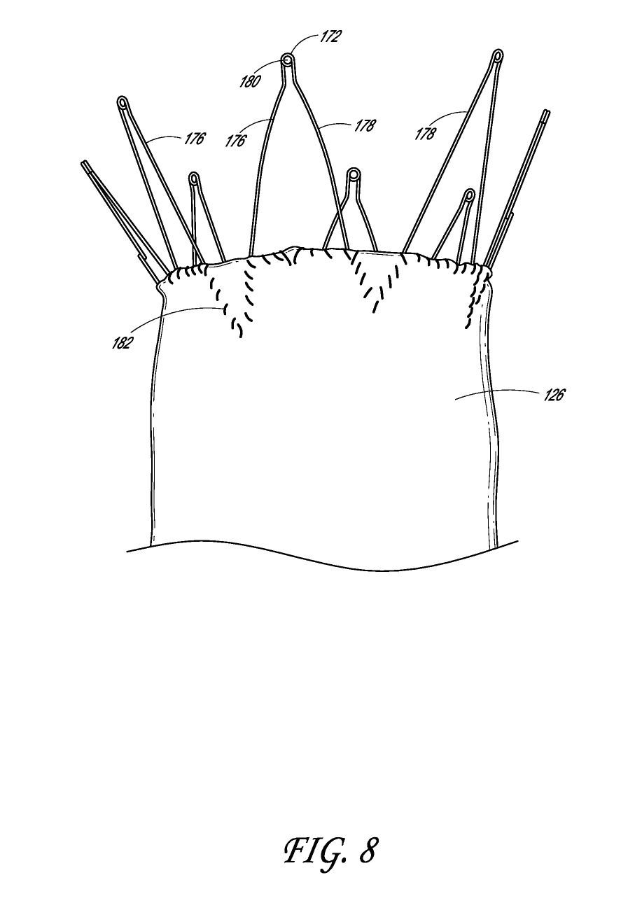

In the illustrated embodiment of FIGS. 8-11, the stent 150 and the segment 170 can have eight distal apices and eight proximal apices. In some embodiments, the stent graft segments 150, 170 can have from six to ten proximal and distal apices, or from seven to nine proximal and distal apices, or any suitable number of proximal and distal apices.

In some embodiments, the longitudinal length of the exposed wire portion of the segment 170 (i.e., beyond the distal end of the graft 154) or any partially uncovered segment disclosed herein can be approximately 20 mm, or from approximately 5 mm to approximately 40 mm, or from approximately 15 mm to approximately 30 mm, or to or from any values within these ranges. Some embodiments of the graft 154 can terminate adjacent to the proximal end 166 to expose at least a portion of the segment 170 and extend over the full length of the first proximal segment 156. In some embodiments, a proximal portion of the first proximal segment 156 and a portion of the segment 170 can be exposed.

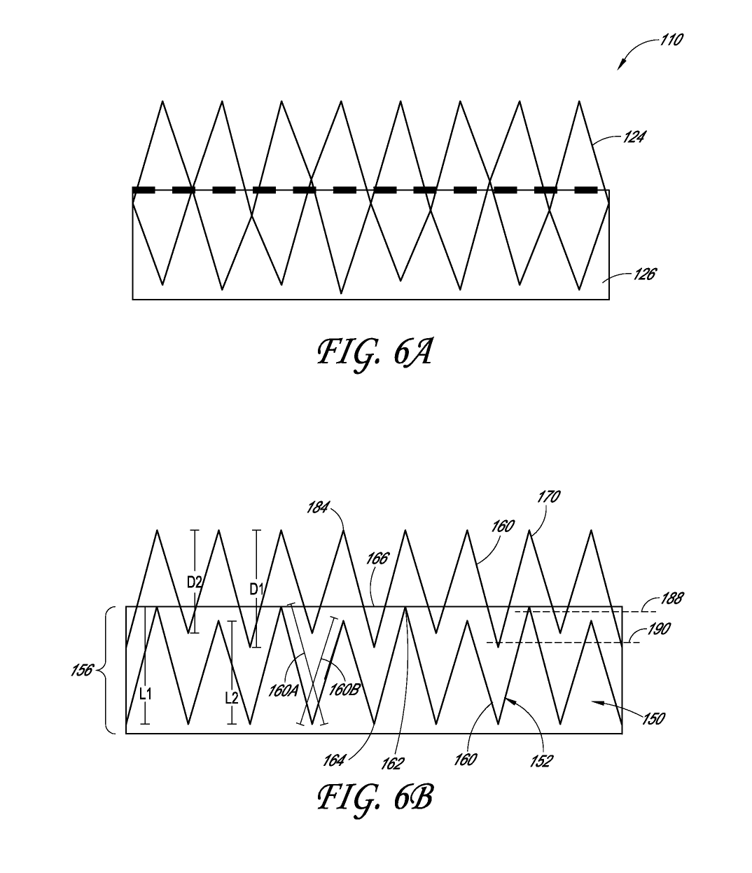

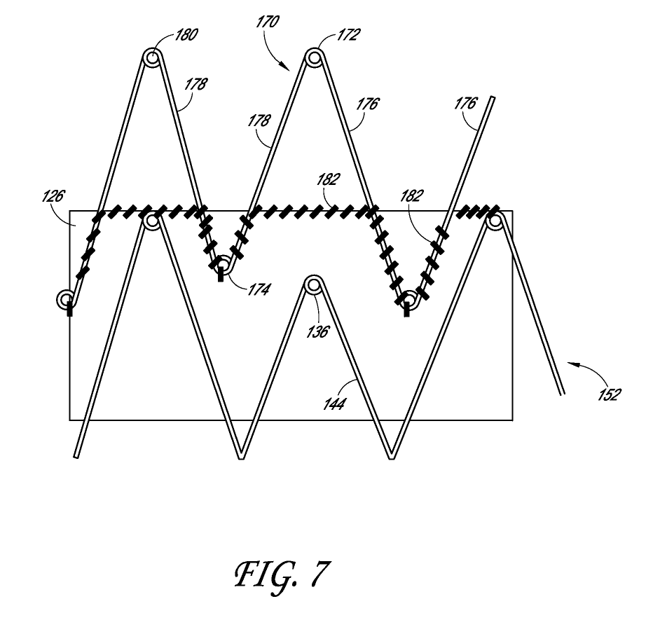

As illustrated in FIGS. 6B and 7, the first proximal segment 156 can have a plurality of proximal apices 162. The longitudinal length of the proximal apices 162 can vary from a first apex 162 having a length L1 (along a line parallel to the axial centerline of the stent 150) to an adjacent second apex 162 having a length L2 (along a line parallel to the axial centerline of the stent 150). The variation of the longitudinal length of the apices occurs intermittently, alternating between L1 and L2 from one apex to the next in a circumferential direction of the stent 150. In some embodiments, in this configuration, no two adjacent apices 162 will have the same longitudinal length or longitudinal (axial) position. In some embodiments, the intermittent variation in the longitudinal length of the apices 162 can occur every third or fourth apex, or the like, such that two or three circumferentially adjacent apices 162 can have the same longitudinal length L1 followed by an adjacent apex 162 with a different longitudinal length L2. In some embodiments, the apices can have more than two differing longitudinal lengths, e.g. L1, L2, L3, L4, or the like such that the apices 162 occupy any of two, three, four, or more longitudinal (axial) positions. In some embodiments, the intermittent variation in longitudinal length can be in any desired order or sequence, or alternatively in any random sequence, around the circumference of the stent 150. In some embodiments, the proximal apices 162 can have approximately the same longitudinal length.

The variation in longitudinal lengths of the proximal apices 162 can result in the proximal apices 162 of the stent segment 156 being longitudinally staggered, such that not all proximal apices 162 are lying on a common plane that is perpendicular the longitudinal axis. Accordingly, some of the proximal apices 162 (for example, without limitation, every second or third proximal apex 162) of the stent segment 152 can be positioned approximately on a first axial plane 188, and some of the proximal apices 162 (for example, without limitation, the proximal apices 162 not positioned approximately on the first axial plane 188) can be positioned approximately on a second axial plane 190.

Similarly, with reference to FIG. 6B, some of the distal apices 174 of the stent segment 170 (for example, without limitation, every second or third distal apex 174) can be positioned approximately on a third axial plane 191, and some of the distal apices 174 (for example, without limitation, the distal apices 174 not positioned approximately on the third axial plane 191) can be positioned approximately on a fourth axial plane 193 or on another axial plane (not illustrated).

As illustrated in FIG. 6B, the struts 160 of the stent segment 156 can have one of two or more different lengths 160A, 160B. The struts of the stent segment 170 can similarly define two or more different lengths. The staggered, or intermittent, longitudinal lengths of the proximal apices 162 can form a W-shaped pattern 204, as depicted in FIG. 10, similar to an M-shaped pattern 202 as described in detail below.

In any of the embodiments disclosed herein, the lengths of the struts of the stents or stent segments can be the same or approximately the same (within 10%-15% of one another). For example, with respect to the stent segment 156, the lengths 160A, 160B of the struts 160 can be the same or approximately the same (within 5%-15% of one another) in some embodiments. The same configurations can apply to the struts of the stent segment 170, or any other stent disclosed herein. In the embodiments where the struts have the same or approximately the same lengths, the angles between the adjacent struts can vary from one to the next to accommodate the pattern of the struts. Forming stents with the same or approximately the same strut length can improve the flexibility characteristics of the stent.

In some embodiments, the stent segments can be configured to define the same or approximately the same (within 5%-15% of one another) angle between each of the struts. In some embodiments, the stent segments can be configured to define a varying angle between one or more of the struts. The apices can be equally spaced about the circumference of the stent graft, or can define varying distances between each of the apices.

The first proximal segment 156 further can have a plurality of distal apices 164 that define the distal (i.e., downstream) end of the proximal segment 156. In some embodiments, the distal apices 164 can lie on a common plane that is perpendicular to the longitudinal axis of the support 152. In some embodiments, the distal apices 164 can have a plurality of longitudinal lengths or positions that vary in a similar manner as described above for the proximal apices 162.

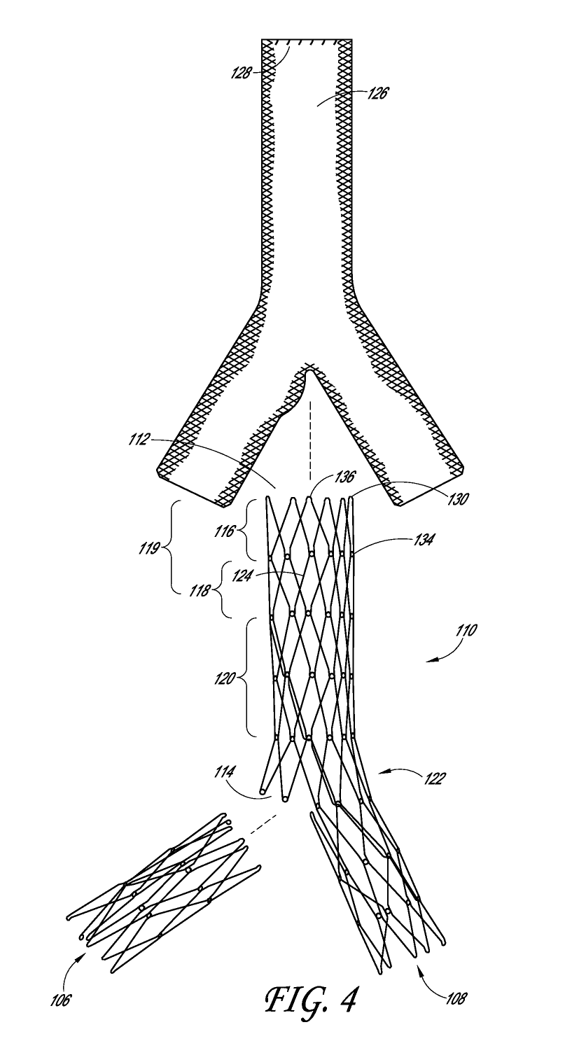

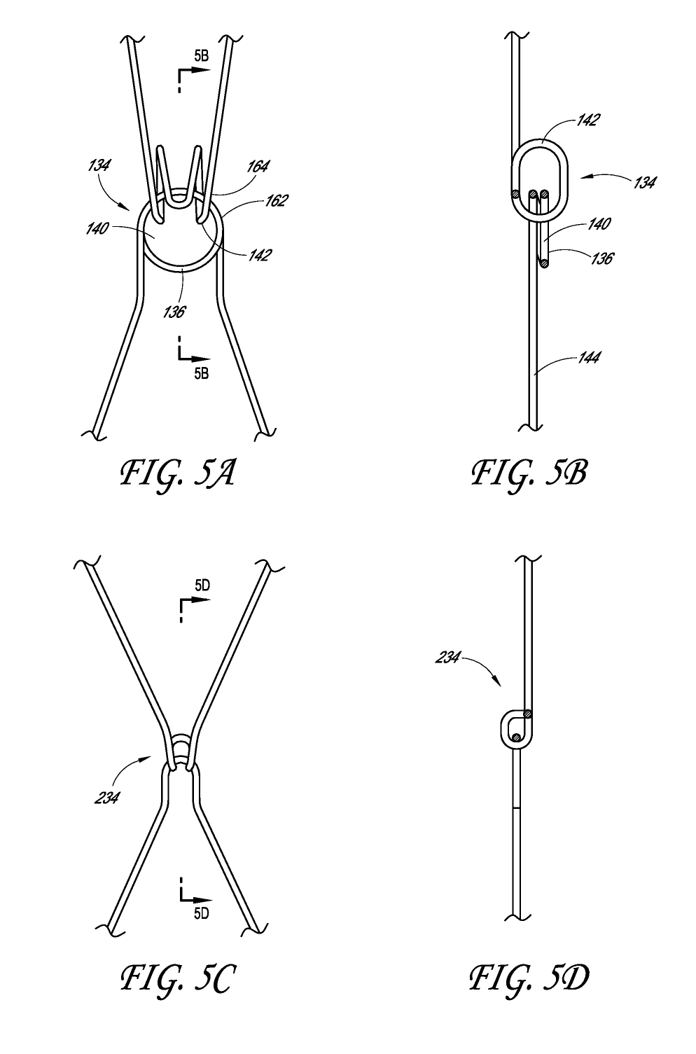

In some embodiments, the first proximal segment 156 can be coupled to the second proximal segment 158 in a manner similar to that described above for the main graft body 110. With reference to FIG. 5, the first proximal segment 156 and the second proximal segment 158 can be coupled together by links 134 or links 234. The links 134 can couple the plurality of distal apices 164 with the plurality of proximal apices 162. In some embodiments, the distal apices 164 can each define a hook 142 that lockingly engages the loop 136 of a proximal apex 162. The locking engagement occurs via the hook 142 passing through a first aperture 140 defined by the loop 136. Any of the apices disclosed herein can comprise links, bends, loops, or any of the other features disclosed or incorporated by reference herein. Therefore, even though some of the illustrations show the apices having particular features, such as loops, links, bends, or otherwise, it is to be understood that such apices can have any suitable features alternatively to or in addition to those shown in the illustrations.

In some embodiments, as illustrated in FIGS. 3 and 7-11, the segment 170 can have a proximal apex 172, a distal apex 174, a first strut 176, and a second strut 178. The proximal apices 172 can define the proximal end of the stent segment 170, and the distal apices 174 can define the proximal end of the independent segment 170. The first strut 176 and the second strut 178 can connect circumferentially adjacent proximal and distal apices. The independent segment 170 can have a substantially repeating pattern of a proximal bend or apex 172, connected via the first strut 176 to a corresponding distal bend or apex 174, which in turn can be connected via the second strut 178 to an adjacent proximal bend or apex apex 172. The pattern can extend in a generally zig-zag configuration around the circumference of the stent 150. The apices of any of the embodiments disclosed herein can take the geometric form of any of a variety of type of bends, e.g. a loop, annulus, eye, a U-shaped bend, any acute angle, a combination of independent or distinct bends, or the like.

In a manner similar to the proximal apices of the first proximal segment 156, a plurality of distal apices 174 can be positioned adjacent to different planes and can alternate intermittently about the circumference of the stent 150. As such, with reference to FIG. 6B, the longitudinal length of the distal apices 174 can vary from a first apex 174 having a length D1 to an adjacent second apex 174 having a length D2. In some embodiments, the apex 174 longitudinal length variation can occur intermittently, alternating between length D1 and D2 from one apex to the next circumferentially around the stent 150 and segment 170, such that no two adjacent distal apices 174 are positioned on the same plane.

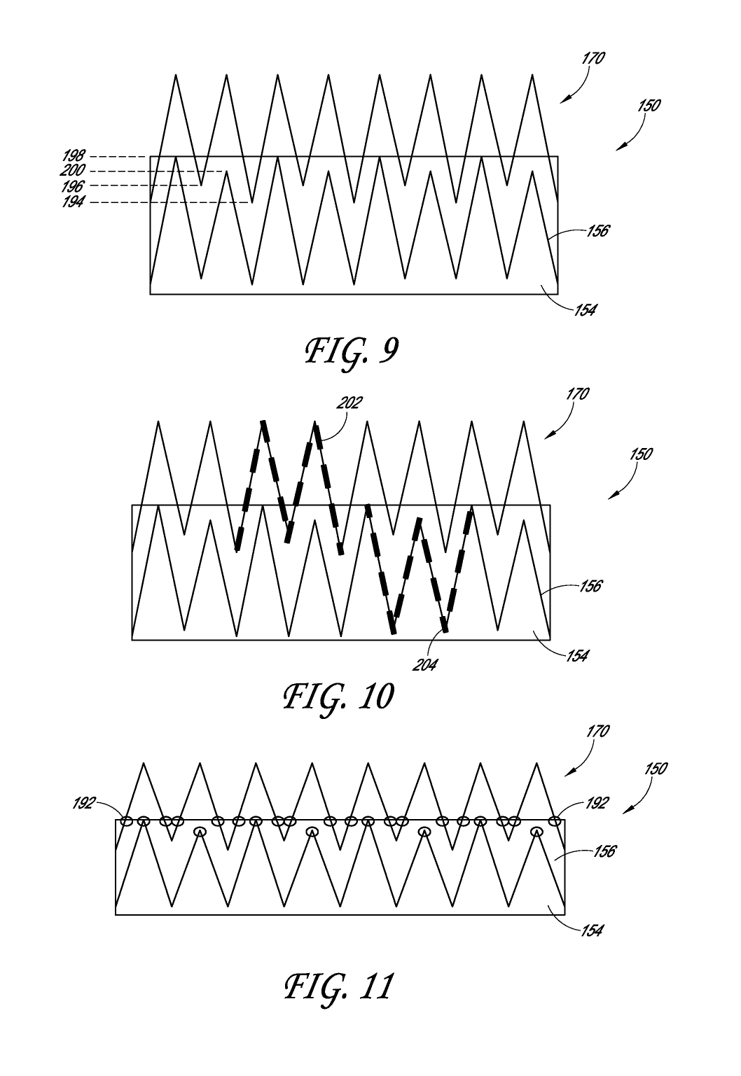

As shown in FIG. 7, the distal apices 174 can be staggered about a apex plane 195 located longitudinally between the apex lengths D1 and D2, just as proximal apices 162 can be staggered about apex plane 197. In some embodiments, the distal apices 174 can include any number of different lengths for an individual distal apex 174, e.g. D1, D2, D3, D4, D5, and the like. In some embodiments, the distal apices 174 having differing longitudinal lengths can be arranged in any order or sequence about the circumference of the stent 150. The two different longitudinal lengths of the distal apices 174 can result in the apices lying on one of two or more (two being shown) different perpendicular planes 194, 196, as shown in FIGS. 7 and 9. The staggered, or intermittent, longitudinal lengths of the proximal apices can form an M-shaped pattern 202, as depicted in FIG. 10, similar to the W-shaped pattern 204 as described above.

The intermittent variation in the longitudinal lengths of the distal apices 174 of the independent segment 170 and the distal segment 154 of the extension body 150 can provide a significantly increased number of contact points 192 with the aorta 10 vessel wall, as illustrated in FIG. 11. For example, FIG. 11 illustrates a total of 24 separate and discrete (i.e., spaced apart) contact points in the distal seal zone. By comparison, when facing apices adjacent the distal end of the sleeve 126 are mechanically connected, as illustrated in FIG. 6A, the number of contact point can be significantly reduced. The sealing function of the intermittent longitudinal lengths and the circumferentially offset apices can be further increased because the apices of the proximal segment 156, or the infrarenal segment, can move radially independently of the segment 170. The independent radial movement of the two segments adjacent the distal end of the stent 150 provides increased conformity of the distal seal zone with the irregularities along the aorta 10 vessel wall.

The variation in the longitudinal lengths of the distal apices 174 can reduce the thickness or profile of the segment 170 because the bends of the proximal apices are not all positioned on the same perpendicular plane relative to the longitudinal axis of the extension body 150, and because there are no direct apex to apex connections between the distal apices 174 of the segment 170 with the proximal apices 162 of the stent segment 156. The varying longitudinal lengths of the distal apices 174 can establish a longitudinal offset between the bends of the distal apices 174 such that only a subset of the apices, if any, are aligned about a plane perpendicular to the longitudinal axis. The reduced number of apices at such a plane defines a much smaller cross-sectional diameter for the proximal portion of the stent 150, potentially enabling a smaller diameter deployment catheter to be used. In some embodiments, the deployment catheter used to deploy the stent 150 can have a diameter less than 21 Fr., permitting better clearance and less trauma within the patient's vasculature during deployment.

In some embodiments, the distal apices 174 can be substantially equally spaced about the circumference of the stent 150, resulting in substantially equal arc lengths between adjacent distal apices 174. In some embodiments, the distal apices 174 can have different arc lengths between adjacent apices to establish local offset relationships between the distal apex 174 and a substantially circumferentially aligned geometric feature of the first proximal segment 156 of the extension graft support 152, e.g. a distal apex 162, a strut 160, a proximal apex 164, a bend anywhere therebetween, or the like.

The various segments that make up the wire body support 152 of stent 150 can be connected to one another via the facing apices, i.e. the facing proximal apices and distal apices of two adjacent segments. The proximal apices can be mechanically coupled to circumferentially aligned distal apices of an adjacent segment of the support 152 via the links 134, links 234, or by other suitable means.

For the segment 170, however, in some embodiments, the stent segment 170 can be configured such that none of the distal apices 174 are directly coupled with apices of the segment 156. In this configuration, the segment 170 can be configured to be coupled only directly to the graft 154 and not to the support 152. The portion of the independent segment 170 distal apices 174 that are covered by the graft 154 can be coupled via suture 182 stitching around the wire body to the graft 154 material (FIG. 7). The suture 182 can be stitched through the graft 154 material on opposing sides of the struts 176, 178 as the suture is spirally wound, or interwoven, around the wire body portions.

In the illustrated embodiment of FIGS. 6B and 7, the segment 170 can be coupled to the graft 154 in a circumferential offset relationship relative to the segment 156 such that the between distal apices 174 of the segment 170 and the proximal apices 162 of the segment 156 are circumferentially offset. As illustrated, in some embodiments, the apices of the segment 170 can be interdigitated with respect to the apices of the segment 156 such that at least some of the distal apices 174 of the segment 170 can be positioned distally relative to at least some of the proximal apices 162 of the segment 156. The independent segment 170 and the first proximal segment 156 can be longitudinally arranged such that a substantially perpendicular plane defined by the longest, or distal-most, distal apices 174 of the independent segment 170 can be located distally of the substantially perpendicular plane defined by the longest, or proximal-most, proximal apices 162 of the first proximal segment 156. In other words, the opposing protruding apices can longitudinally overlap each other, such that at least some of the proximal apices 170 are located between adjacent struts 160. The amount of the interdigitation (i.e., the longitudinal overlap) can be varied to minimize the number of apices that are co-located on the same perpendicular plane, again to minimize the collapsed, pre-deployment diameter or cross-sectional profile of the stent 150.

The selective location of independent segment 170 and the distal end of the graft 154 terminating at the distal-most distal apices 162 can facilitate better visualization of the support 154 under fluoroscopy. Increased visualization provides greater certainty as to where the graft 154 material ends and the suprarenal independent segment 170 begins, which can be critical to preventing obstruction of the renal arteries while at the same time providing sufficient longitudinal length for suprarenal location of the bare wire portion of the independent segment 170. By comparison, in some embodiments, where the independent segment 170 is coupled to the first distal segment 162 via links 134, it can be difficult to visualize the location of the graft 154. The difficulty can arise because the proximal apices 162 of the first proximal segment 156 are not aligned with the proximal end of the graft 154 material when the graft 154 is located to establish the approximately 20 mm of bare wire exposed for suprarenal orientation.

In any of the embodiments disclosed herein, one or more radiopaque markers can be supported by the stent and/or graft or cover of the prosthesis to improve visualization under fluoroscopy. For example, in some embodiments, the stents or stent segments can be sutured to the graft material using radiopaque material, or the graft or cover can otherwise comprise radiopaque sutures to aid in the visualization under fluoroscopy. Additionally, the proximal struts or portions of the proximal stent or stent segment can comprise radiopaque materials or markers to aid in visualization.

In some embodiments, the segment 170 can be fabricated with different or varying characteristics and properties compared to the support 152. In some embodiments, the cross-section of the wire forming the independent segment 170 can be selectively tapered or varying to define selective expansion loads, directions, and geometry. For example, the proximal apices 172 can target a selective outwardly flared maximum diameter to optimize the suprarenal fixation between the distal segment 170 and the aorta 10 native vessel wall. In some embodiments, the circumferential spacing of the distal apices 174 or the proximal apices 172 can be irregular, rather than equidistant between apices. In some embodiments, the number of different lengths for the distal apices 174 can be greater than, less than, or equal to the number of different lengths of the first proximal segment 156 of the support 152. In some embodiments, the expansion loads and the resultant expanded diameter of the distal apices 174 can be greater than the expansion loads and the resultant expanded diameter of the first proximal segment 156 to urge the graft 154 to locally expand radially outward more in the region adjacent the renal arteries. A larger expanding independent segment 170 can be assembled in conjunction with a graft 154 having an expanding tapered distal end to provide a larger diameter adjacent the infrarenal portion of the aorta 10.

In the illustrated embodiment of FIG. 7, a suture stitching is shown that couples the segment 170 to the graft 154. The suture can be spirally wrapped around the wire body contacting the sleeve 153. In some embodiments, the suture can be wrapped through a proximal loop 180 if the segment apices include such a loop feature. The suture can be stitched along the distal end of the graft 154 upon reaching the intersection of a first strut 176 and the graft 154 distal end. The suture stitching can approach an adjacent second strut 178 and can transition from stitching the graft 154 to spirally wrapping the second strut 178. The suturing region transition from the strut to the sleeve and back to the strut can continue around the full circumference of the stent 150 such that there can be a continuous suture coupling the independent segment 170 to the graft 154. In some embodiments, the suture stitching can be spirally wrapped around only the overlapping wire body portions of the independent segment 170 and the graft 154. In some embodiments, a plurality of suture portions can be spirally wrapped around the wire body portions rather than a single continuous suture stitch. In some embodiments, the suture can be stitched, or interwoven, in any direction sufficient to maintain the coupling to the graft 154 and prevent migration within the blood vessel.

The various wire body features such as bend sizing, strut lengths, angles, materials, and material properties can be varied for the fabrication of the independent distal segment as described in the aforementioned references that are incorporated by reference herein, as described above. The neck diameter of the endoluminal prosthesis, or more particularly, the stent 150 can be generally within the range of approximately 10 mm to approximately 40 mm, or from approximately 15 mm to approximately 35 mm, or from approximately 18 mm to approximately 32 mm.

The stent 150 outer diameter can be within the range of approximately 10 mm to approximately 50 mm, or from approximately 20 mm to approximately 40 mm, or from approximately 25 mm to approximately 34 mm. The stent 150 longitudinal length can be from approximately 50 mm to approximately 150 mm, or from approximately 65 mm to approximately 130 mm, or from approximately 75 mm to approximately 120 mm. The longitudinal length of the graft 154 covered portion of the stent 150 can be from approximately 30 mm to approximately 130 mm, or from approximately 45 mm to approximately 120 mm, or from approximately 55 mm to approximately 100 mm. The diameter of the delivery catheter for the endoluminal prosthesis can be from approximately 15 Fr to 25 Fr, or from approximately 18 Fr to approximately 21 Fr, or from approximately 19 Fr to approximately 21 Fr.

In some embodiments, a segment such as the segment 170 can be coupled to the sleeve 126 of the main graft 110. The ability to locate the distal end 112 of the main body 110 in longitudinally short infrarenal portions of the aorta 10, or to locate a longer straight portion of the main body 110, can provide capability to deploy the main graft 110 adjacent the renal arteries 12, 14, or any other vessel branches, and avoid blood flow blockage or other obstruction of the arteries. In some embodiments, the segment 170 can be incorporated in any stent graft, on either a proximal or a distal side. Additionally, in some embodiments, a proximal or distal end portion of any suitable stent grafts can be configured to support a stent segment having any of the features of the stent segments 156, 170, which can improve the sealing function of the stent and/or reduce the profile thickness of the end portion(s) of the stent.

The endoluminal prosthesis can be well suited for use in abdominal aortic aneurysm repair procedures. During the endovascular abdominal aortic aneurysm repair procedure, the self-expanding bifurcated stent graft can be deployed by means of the 21 F catheter delivery system. The effect of the deployed stent graft can be to exclude the aneurysm. The delivery system can be designed to provide accurate positioning of the stent graft, or endoluminal prosthesis, during delivery with minimally invasive, or femoral, access to the body. The delivery system also allows for readjustment during the delivery of the stent graft.

Any portion of the device 100, including the stents 156, 170, can be formed from any of a variety of biologically compatible materials, e.g. metal alloys such as elgiloy, nitinol, or other alloys which include nickel, titanium, tanatalum, stainless steel, or the like, or any combination thereof. Further, the wire size, or gauge, the shape, and the material can be varied to change the elasticity, structural rigidity, and expanded shape of the wire support 124 and the stent 150. Finally, the device 100 can have any of the other details or features of any of the embodiments of the stents disclosed in U.S. Pat. No. 6,733,523, U.S. Pat. No. 6,077,296, and/or U.S. Provisional Patent Application No. 61/231,898, each of which is hereby incorporated by reference as if fully set forth herein.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. For example, the embodiments disclosed above can be used to repair vasculature in other portion of the body, including but not limited to the superior mesenteric artery, the inferior mesenteric artery, or any other arteries or blood vessels in the body suitable for such procedures or apparatuses.

In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments can be made and still fall within the scope of the invention. Accordingly, it should be understood that various features and aspects of the disclosed embodiments can be combine with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. An endoluminal prosthesis comprising:
a graft having a first end and a second end;
a first stent positioned at a first end of the graft, the first stent comprising a plurality of proximal apices and a plurality of distal apices; and
a second stent positioned axially adjacent to the first stent comprising a plurality of proximal apices positioned at a first end of the second stent;
wherein:
the first stent is partially covered by the graft such that the proximal apices of the first stent are not covered by the graft;
one or more of the distal apices of the first stent is positioned approximately on a first plane that is perpendicular to a longitudinal axis of the first stent;
one or more of the distal apices of the first stent is positioned approximately on a second plane that is perpendicular to a longitudinal axis of the first stent, the second plane being offset from the first plane;
the second stent is positioned relative to the first stent such that the plurality of proximal apices of the second stent are spaced apart from the plurality of distal apices of the first stent;
one or more of the proximal apices of the second stent is positioned approximately on a third plane that is perpendicular to a longitudinal axis of the second stent, the third plane being offset from the first and second planes;
one or more of the proximal apices of the second stent is positioned approximately on a fourth plane that is perpendicular to a longitudinal axis of the second stent, the fourth plane being offset from the first, second, and third planes; and
the distal apices of the first stent are circumferentially offset from the proximal apices of the second stent.

2. The endoluminal prosthesis of claim 1, wherein the second stent comprises multiple, interconnected axially adjacent segments or rings.

3. The endoluminal prosthesis of claim 1, wherein the second stent is longer than the first stent.

4. The endoluminal prosthesis of claim 1, wherein the apices comprise at least one of a bend, a loop, an annulus, and a U-shaped bend.

5. The endoluminal prosthesis of claim 1, wherein the first and second stent are attached to the graft using sutures.

6. The endoluminal prosthesis of claim 5, wherein at least some of the sutures are radiopaque.

7. The endoluminal prosthesis of claim 1, further comprising one or more radiopaque markers supported by at least one of the graft, the first stent, and the second stent.

8. The endoluminal prosthesis of claim 1, wherein the first stent comprises an M-shaped pattern, and the second stent comprises a W-shaped pattern.

9. The endoluminal prosthesis of claim 8, wherein the first stent comprises struts between each of the apices, and the lengths of each strut is within 5%-10% of the length of every other strut of the first stent.

10. The endoluminal prosthesis of claim 1, wherein the second stent is formed from a continuous length of wire and comprises a plurality of axially adjacent, tubular stent segments.

11. An endoluminal prosthesis comprising:
a first support having a proximal end and a distal end;
a second support spaced apart from the first support having a proximal end and a distal end, the second support being located closer to a distal end of the prosthesis as compared to the first support; and
a cover that at least substantially covers the second support and at least a portion of the first support;
wherein:
at least a portion of each of the first and second supports is attached to the cover;
the first support comprises distal apices that are longitudinally and circumferentially offset as compared to proximal apices of the second support, the distal apices being interdigitated relative to the proximal apices of the second support.

12. The endoluminal prosthesis of claim 11, wherein the distal apices of the first support are configured such that a first distal apex of the first support has a first longitudinal length and a second distal apex has a second longitudinal length, the first longitudinal length being different than the second longitudinal length.

13. The endoluminal prosthesis of claim 11, wherein the proximal apices of the second support are configured such that a first proximal apex of the second support has a first longitudinal length and a second proximal apex has a second longitudinal length, the first longitudinal length being different than the second longitudinal length.

14. The endoluminal prosthesis of claim 11, wherein the distal apices of the first support are coupled to the sleeve via suture stitching.

15. The endoluminal prosthesis of claim 11, wherein the proximal apices of the second support are coupled to the sleeve via suture stitching.

16. A method of deploying an endoluminal prosthesis in vasculature, comprising:
inserting an endoluminal prosthesis within the vasculature, the endoluminal graft having a first support and a second support coupled to a sleeve, wherein the first support is coupled to a proximal end of the sleeve and extends longitudinally beyond the proximal end of the sleeve;
positioning the endoluminal prosthesis adjacent a vascular branch blood vessel by locating a proximal end of the second support adjacent the branch opening, the proximal end of the second support coinciding with the proximal end of the sleeve; and
expanding the endoluminal prosthesis;
wherein:
the first support spans the vessel branch to make contact with the vessel wall on the opposing side of the vessel branch;
one or more distal apices of the first support are longitudinally offset from other distal apices of the first support;
one or more distal apices of the second support are longitudinally offset from other distal apices of the second support; and
each of the distal apices of the second support is circumferentially offset from each of the proximal apices of the first support.

17. The method of deploying an endoluminal prosthesis of claim 16, wherein the adjacent proximal apices of the second support and the distal apices of the first support are interdigitated.
